(19) United States
(12) Patent Application Publication
(10) Pub. No.: US 2025/0331793 A1
(43) Pub. Date: Oct. 30, 2025

(54) METHOD FOR CHECKING A ROTARY BEARING OF A GANTRY OF A COMPUTED TOMOGRAPHY SYSTEM

(71) Applicant: Siemens Healthineers AG, Forchheim (DE)

(72) Inventors: Florian DIETRICH, Forchheim (DE); Klaus MAISEL, Bindlach (DE); Michael HOFMANN, Goldkronach (DE); Mathias HARTMANN, Bad Berneck (DE); Daniel ROEDEL, Bayreuth (DE)

(73) Assignee: Siemens Healthineers AG, Forchheim (DE)

(21) Appl. No.: 19/191,179

(22) Filed: Apr. 28, 2025

(30) Foreign Application Priority Data
Apr. 29, 2024 (DE) 10 2024 203 962.4

(51) Int. Cl. A61B 6/00

(52) U.S. Cl. CPC A61B 6/4447

(57) ABSTRACT

A method for checking a rotary bearing of a gantry of a computed tomography system, comprises: rotating a bearing ring about an axis of rotation relative to a supporting structure, wherein at least two structure-borne sound sensors are attached to the gantry; capturing structure-borne sound values via the at least two structure-borne sound sensors while the bearing ring rotates about the axis of rotation relative to the supporting structure; and evaluating the structure-borne sound values to determine whether interference noise beyond a specified interference noise tolerance occurs when the bearing ring rotates.

### CROSS-REFERENCE TO RELATED APPLICATION(S)

The present application claims priority under 35 U.S.C. § 119 to German Patent Application No. 10 2024 203 962.4, filed Apr. 29, 2024, the entire contents of which are incorporated herein by reference.

### FIELD

One or more example embodiments of the present invention relate to a method for checking a rotary bearing of a gantry of a computed tomography system, a method for evaluating structure-borne sound values for the purpose of checking a rotary bearing of a gantry of a computed tomography system, and a computer program product.

### BACKGROUND

Independent of the grammatical term usage, individuals with male, female or other gender identities are included within the term.

Third-generation computed tomography devices (CT devices) comprise a gantry with an x-ray source and an x-ray detector which are typically embodied to rotate around an examination region at relatively high speed during a CT scan. It is typically the case that rotary bearings do not always behave identically with regard to their noise emission during operation. It is desirable during the manufacture and configuration of a computed tomography device (CT device) to ensure that in subsequent operation the CT device does not itself emit distinct and loud noises, also referred to in the following as interference noise.

In order to capture interference noise at an early stage in the context of quality assurance, the rotary bearings can be measured by picking up an airborne sound during rotation, for example during assembly in the gantry.

It is however typically the case that such sound measurements can be greatly influenced by ambient noise, and this can significantly hamper a precise measurement. For example, it is often difficult and complicated, in a production hall where other assembly tasks are typically in progress at the same time, to avoid external interference noise which can influence the measurement of the rotary bearing.

Furthermore, the measurement itself is usually very complex and requires a precise procedure which may require a certain period of training for relevant personnel, and an incorrect procedure can quickly result in false measurements. A dependency on the position of the gantry in the room and the presence of further objects can also influence the measurement result, in particular due to reflections.

One approach for reducing the influence of ambient noise could be to interrupt other tasks in the surrounding area, for example in a production hall. A measurement could also be taken outside of normal work hours. However, these approaches are often unable to ensure the complete avoidance of ambient noise, and are also usually associated with greater expense and reduced work efficiency.

### SUMMARY

An object of one or more embodiments of the present invention is therefore to provide a mechanism whereby interference noise of a gantry can be identified as reliably as possible, and in particular at an early stage. It is also desirable to allow the detection of interference noise to be as far as possible comparable and reproducible.

At least this object is achieved by methods and non-transitory computer program products as claimed in the independent claims. The dependent claims specify further advantageous aspects of embodiments of the present invention.

An embodiment of the present invention relates to a method for checking a rotary bearing of a gantry of a computed tomography system, the gantry having a supporting structure and the rotary bearing with a bearing ring, the bearing ring being rotatably mounted about an axis of rotation relative to the supporting structure, said method comprising:
rotating the bearing ring about the axis of rotation relative to the supporting structure, at least two, in particular precisely three, structure-borne sound sensors being attached to the gantry, in particular to a surface of the gantry,
capturing structure-borne sound values via the at least two structure-borne sound sensors while the bearing ring rotates about the axis of rotation relative to the supporting structure,
evaluating the structure-borne sound values, in particular with regard to a volume of a structure-borne sound, it being determined whether interference noise beyond, in particular above, a specified interference noise tolerance occurs when the bearing ring rotates.

The inventive method advantageously presents a mechanism and/or means by which it is possible, largely regardless of surroundings, to detect interference noise of the gantry, said noise being caused by rotation of the bearing ring. Given that interference noise is determined, said method is in particular a method for acoustically checking a rotary bearing. It has also been shown that the inventive method can be carried out with great accuracy of repetition, so that in particular effective comparisons can be achieved and corresponding evaluations can be particularly representative. For example, noise-generating activities in the surrounding area, such as assembly tasks related to other devices, do not necessarily have to be interrupted for this method. Furthermore, it has advantageously been established that the inventive method is comparatively economical and can be carried out with relatively little effort in comparison with other measuring methods such as acoustic intensity measurement, for example.

In particular, provision can be made for the gantry to have a drive and/or for the rotation of the bearing ring about the axis of rotation relative to the supporting structure to be driven via the drive. The drive can be a drive which is typically used in computed tomography devices, for example a motor.

In particular, provision can be made for the supporting structure to have a supporting frame and a tilting frame and/or for the tilting frame to be mounted in such a way that it can be tilted about a tilting axis relative to the supporting frame. For example, the tilting frame can comprise the rotary bearing and/or the bearing ring. In particular, the bearing ring can be mounted in such a way that it can be rotated about the axis of rotation relative to the tilting frame. The tilting axis can be horizontal and/or perpendicular to the axis of rotation, for example.

In particular, provision can be made for the bearing ring to be configured for connection to a rotating frame, and in particular such that via the rotary bearing, the rotating frame is mounted together with the bearing ring rotatably about the axis of rotation relative to the supporting structure, in particular relative to the tilting frame, when the connection of the rotating frame to the bearing ring is established. The connection of the rotating frame to the bearing ring can be substantially nondestructively detachable, for example, in particular nondestructively detachable. For example, the rotating frame can be screwed to the bearing ring. Alternatively, provision can be made for the gantry to have a rotating frame, the rotating frame then comprising the bearing ring and/or the bearing ring forming a partial region of the rotating frame.

In particular, provision can be made for the rotating frame to be configured to hold an x-ray source and/or an x-ray detector of the computed tomography system, in particular such that the x-ray source and the x-ray detector are fixed relative to each other when the x-ray source and the x-ray detector are held in the rotating frame.

In particular, provision can be made for the gantry to have a tunnel-shaped opening, said tunnel-shaped opening extending along the axis of rotation. The tunnel-shaped opening can be designed in particular such that an examination object can be introduced into the tunnel-shaped opening along the axis of rotation for examination via the computed tomography system. In particular, provision can be made for the examination object within the tunnel-shaped opening to be stationary relative to the supporting structure during the examination via the computed tomography system, while the bearing ring rotates about the axis of rotation relative to the supporting structure, so that in particular the x-ray source and/or the x-ray detector of the computed tomography system rotate around the examination object. The examination object can be a patient, for example.

The term “bearing ring” has a broad sense in the context of embodiments of the present invention. In particular, provision can be made for the bearing ring to be an annular body and/or for the rotary bearing to be a rolling bearing. In particular, provision can be made for the bearing ring to be mounted in such a way that, with the aid of rolling elements of the rotary bearing, it can rotate about the axis of rotation relative to the supporting structure. In particular, a detrimental mounting of the bearing ring can result in interference noise, which can be determined using this method.

The method for checking a rotary bearing can be in particular a method for checking a rotary bearing during manufacture and/or assembly of the gantry and/or of the computed tomography system. For example, the method can be a method for checking a rotary bearing in the context of batch testing and/or goods inwards testing of the components of the gantry. The x-ray detector and the x-ray source are preferably not yet installed on the bearing ring when the method is carried out. Identification of interference noise can then be carried out at a particularly early stage, even before the installation of these components. It has been shown that subsequent interference noise can already be determined even without installed x-ray source and x-ray detector. It is however fundamentally also conceivable to carry out the method with x-ray detector and x-ray source installed. A brief warm-up phase of the bearing ring is preferably performed in advance of the described method. A new rotary bearing typically requires a certain run-in period first, after which constant sound values can be measured. By virtue of including a warm-up phase, it is possible to achieve measurement results that can be more representative of subsequent behavior during operation. For example, the warm-up phase can take place over a number of minutes, preferably 5 to 45 minutes. However, the method need not necessarily be restricted to a warm-up phase, in particular not to a specific warm-up phase.

The term “structure-borne sound sensor” has a broad sense in the context of embodiments of the present invention. It designates generally a sensor that is configured to capture structure-borne sound values. A structure-borne sound value is understood in particular to be a sound which propagates in a solid. Structure-borne sound values can be understood to be corresponding measured values of a structure-borne sound. In particular, the structure-borne sound sensor can be so embodied as to pick up a vibration on a surface of a body and thus to capture a structure-borne sound value.

According to embodiments of the present invention, at least two structure-borne sound sensors are attached to the gantry, in particular to the supporting structure, for example to the supporting frame and/or the tilting frame. The structure-borne sound sensors are preferably attached to a surface of the gantry, in particular a surface of the supporting structure, for example a surface of the supporting frame and/or a surface of the tilting frame. A different association with the gantry is however also conceivable, for example integration into the gantry, provided that a structure-borne sound can be picked up thereby. An attachment to a surface represents a particularly simple possibility.

While structure-borne sound values can generally also be captured using only one structure-borne sound sensor, it is possible via a plurality of sensors to achieve a measurement result which is clearly more suitable for determining interference noise. At least within certain limits, various points on the gantry typically have different structure-borne sound values, each of which can have an influence on subsequent interference noise or be related to subsequent interference noise. More sensors normally allow more information to be obtained, and therefore a more precise result can be achieved using a larger number of sensors. However, it has been shown that even with three sensors and suitable placement which can be ascertained via test measurements, for example, interference noise can already be ascertained with great precision. It is thereby advantageously possible to dispense with the fitting of further structure-borne sound sensors.

For the measurement of the structure-borne sound values, the bearing ring is preferably rotated at a normal operating speed. It is thereby possible to effect a particularly representative measurement. The structure-borne sound values can be captured in a frequency-dependent manner in particular. The structure-borne sound values can be captured as a velocity level, for example, in particular a mean velocity level. The velocity level can also be referred to as a sound particle velocity level. The velocity level is a logarithmic value derived from the sound particle velocity (also abbreviated as “velocity”). For example, the structure-borne sound can be captured as a mean velocity level in accordance with DIN 45635-8 “Gerauschmessung an Maschinen; Luftschallemission, Körperschallmessung; Rahmenverfahren” (publication date 1985 June).

The volume of the structure-borne sound has a broad sense in the context of embodiments of the present invention. In particular, it is understood to be a variable-related classification of the structure-borne sound. For example, the classification can be made with reference to the mean velocity level. The classification can be based on purely physical variables or on a variable which takes into account the human sound sensation, such as a loudness level, for example. Interference noise resulting from this is rated with reference to a specified interference noise tolerance, by determining whether interference noise beyond, in particular above, the specified interference noise tolerance occurs when the bearing ring rotates. The specified interference noise tolerance can be defined, for example, via a threshold value and/or a reference curve or reference value range.

According to an embodiment variant, the at least two structure-borne sound sensors comprise an acceleration pickup for picking up a structure-borne sound pressure. An acceleration pickup can be particularly suitable for capturing the structure-borne sound values. The at least two structure-borne sound sensors can comprise a piezoelectric element in particular, by which the structure-borne sound is converted into an electric signal. The piezoelectric element can be provided for the purpose of converting dynamic deflections at the surface of the gantry into electric signals. For example, a vibrating mass and a pretension spring which transfer the deflections to the piezoelectric element can be used for this.

According to an embodiment variant, the at least two structure-borne sound sensors are attached to the surface magnetically, by frictional engagement, in particular screw connection, and/or by material engagement, in particular using an adhesive and/or wax. For example, the structure-borne sound sensors can comprise magnets for attachment to the gantry. Provision can optionally be made to use an attachment with a reciprocal magnet on an opposite surface of the gantry. A magnetic attachment can advantageously allow particularly simple and rapid attachment, which is at the same time precise. Wax can be advantageous because it can allow particularly precise fitting and pickup of structure-borne sound.

According to an embodiment variant, the evaluation comprises breaking down the structure-borne sound values into a frequency spectrum. In particular, the determination of interference noise can be carried out in a frequency-dependent manner. The method also comprises performing the assignment directly after or during the measurement. The structure-borne sound values can comprise a measured series of structure-borne sound values which are assigned to a frequency. The frequencies can be sound frequencies in particular. A breakdown into a frequency spectrum can advantageously allow the determination of a particularly good and reliable estimate of subsequently occurring interference noise. For example, the structure-borne sound values can be categorized in a frequency-dependent manner taking the human sound sensation into account.

According to an embodiment variant, a velocity level spectrum is determined. In other words, the breakdown of the structure-borne sound values into a frequency spectrum can include determining the velocity level spectrum. The velocity level spectrum can be based on a mean velocity level in particular. The definition of the mean velocity level is known from the prior art. In particular, the mean velocity level can be determined in accordance with a standard such as DIN 45635-8, for example. It has been shown that the use of a velocity level spectrum, in particular based on the mean velocity level, can deliver particularly accurate and repeatable results for the purpose of determining interference noise. The structure-borne sound sensors can be embodied to pick up a velocity level spectrum, in particular a mean velocity level spectrum.

According to an embodiment variant, mean velocity levels determined in the velocity level spectrum are assigned to specified frequency segments, in particular standard frequency segments. Mean velocity levels determined in the velocity level spectrum are preferably assigned to third-octave center frequencies. Third-octave center frequencies can be a particularly effective mechanism of achieving a precise gradation of the structure-borne sound values and/or breaking down the structure-borne sound values in a frequency-dependent manner. It is thereby advantageously possible to rate every individual third. The structure-borne sound sensors can be embodied to pick up a velocity level spectrum, in particular a mean velocity level spectrum, in specified frequency segments, in particular in third-octave center frequencies. Third-octave center frequencies can be specified in particular according to a specified standard, for example DIN EN ISO 266, “Akustik—Normfrequenzen”, publication date 1997 August.

According to an embodiment variant, determining whether interference noise occurs beyond the specified interference noise tolerance when the bearing ring rotates is performed on the basis of a correlation of the structure-borne sound values, in particular in the form of a frequency spectrum of the structure-borne sound values, with a reference curve. A velocity level spectrum can most preferably be correlated with a reference curve. For the purpose of correlating the structure-borne sound values, a mean value, in particular an energetic mean, can be formed from the at least two structure-borne sound values.

The reference curve can be created at least partially on the basis of empirical values from experts, for example. The reference curve can be created, for example, at least partially on the basis of comparative measurements from gantries, in particular previously tested gantries. The reference curve can allow for typical detected variations in gantries that were tested before and after complete assembly. The reference curve is preferably based at least partially on a measured series of a multiplicity of frequency spectra picked up on gantries, in particular a multiplicity of different gantries. The reference curve does not necessarily have to be embodied as a pictorial curve. The reference curve can optionally be defined by its values, in particular coordinate values, and/or by a curve function. The reference curve can optionally be presented to a user together with the structure-borne sound values via an output medium. The output medium can be a display screen or a printed sheet, for example.

The correlation can comprise, for example, determining whether individual structure-borne sound values of a sound spectrum, in particular a velocity level spectrum, exceed the values of the reference curve. For example, a quantity can be calculated for an excessiveness of individual structure-borne sound values. The excessiveness of individual structure-borne sound values can be determined in a frequency-dependent manner. In particular, the excessiveness of individual structure-borne sound values can be determined as a function of specified frequency segments, in particular third-octave center frequencies. The quantity for an excessiveness can be based on the difference between the excessive sound value and the respective value of the reference curve. The difference can be a difference level, for example. The difference, in particular the difference level, can be a quantity for an interference noise. For example, the quantity for an excessiveness can be defined by a relative value which is based on the difference between the excessive sound value and the respective value of the reference curve. The relative value can be a percentage and/or a ratio, for example.

The determination of whether interference noise beyond the specified interference noise tolerance occurs when the bearing ring rotates can comprise combining, in particular adding, a plurality of quantities for an excessiveness of a plurality of individual structure-borne sound values, in particular all individual structure-borne sound values, in order to determine a total interference noise level. The total interference noise level can be correlated with an interference noise tolerance threshold value, so that a classification of the total interference noise level is performed. The classification can rate an instance of exceeding the interference noise threshold value as an instance of exceeding the specified interference noise tolerance. The classification can rate a total interference noise level which does not reach the interference noise threshold value as a result which is not higher than the specified interference noise tolerance. The classification can optionally comprise further classification categories. For example, one of the classification categories can be the classification “Limit value almost reached”.

By virtue of correlation with a reference curve, various interference noises can advantageously be rated differentially at various frequencies. In particular, provision can be made such that the reference curve does not represent a sharply defined limit, but that a totality of all instances of exceeding the reference curve is rated. For example, a (slight) excessiveness in the case of an individual frequency can still be acceptable if otherwise no (or sufficiently few and slight) further instances of exceeding are present. The reference curve can be considered as a curve for a typical bearing, in particular a typical bearing which exhibits sufficiently low interference noise. The classification can be illustrated in an output via a visible marking, for example a color coding. The color coding can be based on a traffic light system, for example. For example, green can represent “good” while red represents higher than the specified interference noise tolerance. Yellow can optionally represent “Limit value almost reached”. A visible marking can allow a user to understand the result more quickly, for example without the user necessarily having to carefully study and rate numerical values.

According to an embodiment variant, individual instances of exceeding the reference curve are weighted according to their influence on a total interference noise. In this case, a sum of all weighted instances of exceeding is correlated with at least one interference noise threshold value, including an interference noise tolerance threshold value, such that a classification of the sum of all weighted instances of exceeding is performed. An instance of exceeding the interference noise threshold value can be rated as an instance of exceeding the specified interference noise tolerance. It is thereby advantageously possible for higher proportions to be weighted more highly. For example, provision can be made for an energetic addition of instances of exceeding. For example, the structure-borne sound values can be logarithmic values, in particular decibel values. For example, the instances of exceeding can be weighted in such a way that the weighted instances of exceeding describe a relative proportion of the total specified interference noise tolerance. For example, the weighted instances of exceeding can be provided as percentages of the specified interference noise tolerance. However, provision can also be made for another notation, for example as an absolute level. In this case, the specified interference noise tolerance can be a specified maximum level sum, for example.

According to an embodiment variant, the evaluation of the structure-borne sound values comprises a conversion of the structure-borne sound values into anticipated airborne sound values and/or a correlation of the structure-borne sound values with reference values of an airborne sound measurement. It has advantageously been shown that the structure-borne sound values can be representative of airborne sound values. Via a correlation or conversion into airborne sound values, it is possible in particular to determine direct information about interference noise that will subsequently be audible via airborne sound.

According to an embodiment variant, the at least two structure-borne sound sensors are attached at measuring points whose positions are adapted for good comparability of the structure-borne sound values with the reference values of the airborne sound measurement. Additionally or alternatively, the measuring points are specifically set in such a way that they correspond to the reference measuring points which were used to ascertain the reference curve.

Most preferably at least three, ideally precisely three, structure-borne sound sensors are attached to the gantry. At least three structure-borne sound sensors have the advantage that, as has been shown, sufficient comparability with the airborne interference noise can be established. It has been found that as a rule, i.e. in all tests, precisely three structure-borne sound sensors are sufficient in this regard provided suitable measuring positions are chosen. Moreover, by virtue of the relatively small number of only three structure-borne sound sensors, the expense in terms of both attachment and cost is relatively low. For example, two structure-borne sound sensors can be placed on a rear side and one structure-borne sound sensor on a front side of the gantry or vice versa. For example, two structure-borne sound sensors which are attached to the same side of the gantry can be attached to opposite sides of the annular form of the gantry. For example, a structure-borne sound sensor can be offset by 70° to 135°, preferably 80° to 110°, most preferably substantially 90°, relative to a structure-borne sound sensor on the opposite side of the annular form of the gantry.

If use is made of the same measuring points as were used to ascertain the reference curve, particularly good comparability can be achieved. It has been shown possible, with relatively little effort, to place the structure-borne sound sensors, in particular at least three structure-borne sound sensors, in such a way that structure-borne sound values can be determined which correspond very closely to the airborne sound that occurs. The precise placement can depend on the respective gantry or type of gantry. Factors can include the material used or the geometric properties of the gantry. The measuring points can be determined by comparing a measured structure-borne sound spectrum with an airborne sound spectrum of the same gantry. In particular, averaged structure-borne sound values of the structure-borne sound sensors can be used for the comparison.

According to an embodiment variant, for the purpose of the evaluation, the structure-borne sound values are rated and/or A-rated according to a defined human sound sensation. An A-rating is based on an international standard frequency rating curve. It is thereby advantageously possible to take a human sensation of the loudness into consideration.

According to an embodiment variant, for the purpose of evaluating the structure-borne sound values, use is made of a method for evaluating structure-borne sound values as described herein, in particular in the following.

Embodiments of the present invention further relate to a method for evaluating structure-borne sound values for the purpose of checking a rotary bearing of a gantry of a computed tomography system, said method comprising:
establishing a velocity level spectrum, velocity level values, in particular mean velocity level values, being assigned in each case to a frequency value, in particular a third-octave center frequency;
correlating the values of the velocity level spectrum with reference values and registering instances of the reference values being exceeded;
weighting the instances of exceeding the reference values according to their influence on a total interference noise and summing the weighted instances of exceeding;
correlating the sum of all weighted instances of exceeding with at least one interference noise threshold value, including an interference noise tolerance threshold value,
performing a classification of the sum of all weighted instances of exceeding, an instance of exceeding the interference noise tolerance threshold value being rated as an instance of exceeding a specified interference noise tolerance.

All advantages and features of the method for checking a rotary bearing can be transferred analogously to the method for evaluating structure-borne sound values of a rotary bearing and vice versa. The structure-borne sound values can be in particular velocity level values, which are captured in a frequency-dependent manner, for example.

Embodiments of the present invention further relate to a computer program product comprising instructions which, when the program is executed by a computer, cause said computer to execute the steps of the inventive method for evaluating structure-borne sound values for the purpose of checking a rotary bearing of a gantry of a computed tomography system. All advantages and features of the method for checking a rotary bearing and of the method for evaluating structure-borne sound values of a rotary bearing can be transferred analogously to the computer program product and vice versa. For example, the computer program product can be stored on a computer-readable storage medium, in particular a non-volatile storage medium.

A further aspect of embodiments of the present invention is a non-transitory computer-readable storage medium, in particular a non-volatile storage medium, on which is stored the computer program product as described herein. For example, the storage medium can be a hard disk, an SSD, a flash memory, an online server, etc.

All advantages and features of the method for checking a rotary bearing, of the method for evaluating structure-borne sound values of a rotary bearing, and of the computer program product can be transferred analogously to the computer-readable storage medium and vice versa.

All of the embodiment variants described herein can be combined with each other unless explicitly stated otherwise.

### BRIEF DESCRIPTION OF THE DRAWINGS

Examples with features that may assist an understanding of the present invention and/or of the technical problem to be solved are explained in the following with reference to the appended figures.

FIG. 1 shows a flow diagram of a method for checking a rotary bearing of a gantry of a computed tomography system.

FIG. 2 shows a flow diagram of a method for evaluating structure-borne sound values for the purpose of checking a rotary bearing of a gantry of a computed tomography system.

FIG. 3 shows a further example of a flow diagram of a method for checking a rotary bearing of a gantry of a computed tomography system.

FIG. 4 shows an example of the placement of three structure-borne sound sensors on a gantry from a front view.

FIG. 5 shows the example of the placement of the three structure-borne sound sensors on the gantry shown in FIG. 4 from a rear view.

FIG. 6 shows an example of an evaluation of structure-borne sound values.

FIG. 7 shows a further example of an evaluation of structure-borne sound values.

FIG. 8 shows a further example of an evaluation of structure-borne sound values.

FIG. 9 shows a further example of an evaluation of structure-borne sound values.

### DETAILED DESCRIPTION

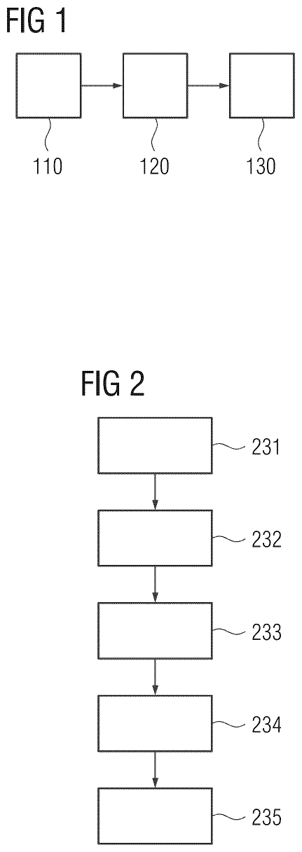

FIG. 1 shows a flow diagram of a method for checking a rotary bearing D of a gantry 1 of a computed tomography system, said gantry 1 having a supporting structure T and the rotary bearing D with the bearing ring L, the bearing ring L being rotatably mounted about an axis of rotation A relative to the supporting structure T, said method comprising:
rotating 110 the bearing ring L about the axis of rotation A relative to the supporting structure T, at least two, in particular precisely three, structure-borne sound sensors 2 being attached to the gantry 1, in particular to a surface of the gantry 1,
capturing 120 structure-borne sound values via the at least two structure-borne sound sensors 2 while the bearing ring L rotates about the axis of rotation A relative to the supporting structure T,
evaluating 130 the structure-borne sound values, in particular with regard to a volume of a structure-borne sound, it being determined whether interference noise beyond, in particular above, a specified interference noise tolerance occurs when the bearing ring L rotates.

As part of the evaluation, the structure-borne sound values can optionally be broken down into a frequency spectrum, so that the determination of interference noise can preferably be carried out in a frequency-dependent manner. In particular, a velocity level spectrum 11 can be determined. The measurement of the structure-borne sound values would already take place in a frequency-dependent manner accordingly.

FIG. 2 shows a flow diagram of a method for evaluating structure-borne sound values for the purpose of checking a rotary bearing D of a gantry 1 of a computed tomography system. In a first step 231, a velocity level spectrum 11 is established. In this case, velocity level values, preferably mean velocity level values 14, are each assigned to a frequency value. The assignment can relate to frequency segments in particular, i.e. the velocity level values of a defined frequency range are each assigned to a frequency value of the frequency range. The frequency value can be in particular a mean frequency value of the frequency range. The frequency value can most preferably be a third-octave center frequency. In a further step 232, the values of the velocity level spectrum 11 are correlated with reference values and instances of exceeding the reference values are registered. In a further step 233, the instances of exceeding the reference values are weighted according to their influence on a total interference noise, and the weighted instances of exceeding 15 are summed. The weighted summing can comprise in particular an energetic addition of the instances of exceeding. For example, relative instances of exceeding in a loud range (for example a high dB(A) value) can be weighted more heavily than instances of exceeding in a quiet range (for example a low dB(A) value). In a further step 234, the sum 16 of all weighted instances of exceeding 15 are correlated with at least one interference noise tolerance threshold value 17. Provision can optionally be made for further interference noise threshold values 18. The further interference noise threshold values 18 can identify various quality grades in respect of the development of interference noise. In a further step, a classification of the sum 16 of all weighted instances of exceeding 15 is performed. In this case, an instance of exceeding the interference noise tolerance threshold value 17 is rated as an instance of exceeding a specified interference noise tolerance.

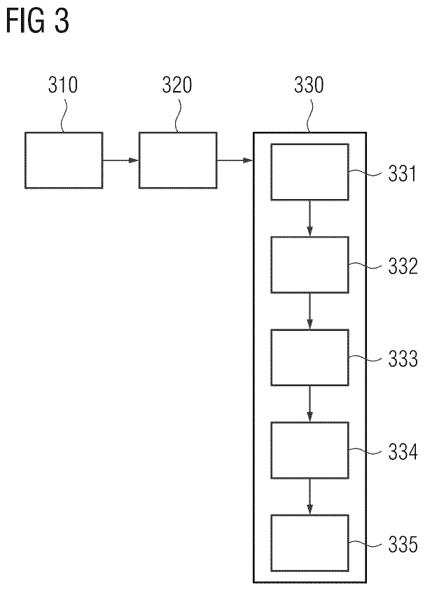

FIG. 3 shows a further example of a flow diagram of a method for checking the rotary bearing D of the gantry 1 of a computed tomography system. In the step 310, the bearing ring L of the gantry 1 is rotated about the axis of rotation A relative to the supporting structure T, at least two, preferably three, structure-borne sound sensors 2 being attached to the gantry 1. In the step 320, during the rotation of the bearing ring L, structure-borne sound values of the bearing ring L are captured by the structure-borne sound sensors 2 attached to the gantry 1. The structure-borne sound values comprise velocity level values, in particular mean velocity level values 14, which are captured in each case with reference to a frequency value, in particular a third-octave center frequency. In the step 330, the structure-borne sound values are evaluated with regard to the volume of the structure-borne sound. The further step 330 comprises a plurality of substeps 331-335, which correspond to the steps 231-235 of the method described with reference to FIG. 2.

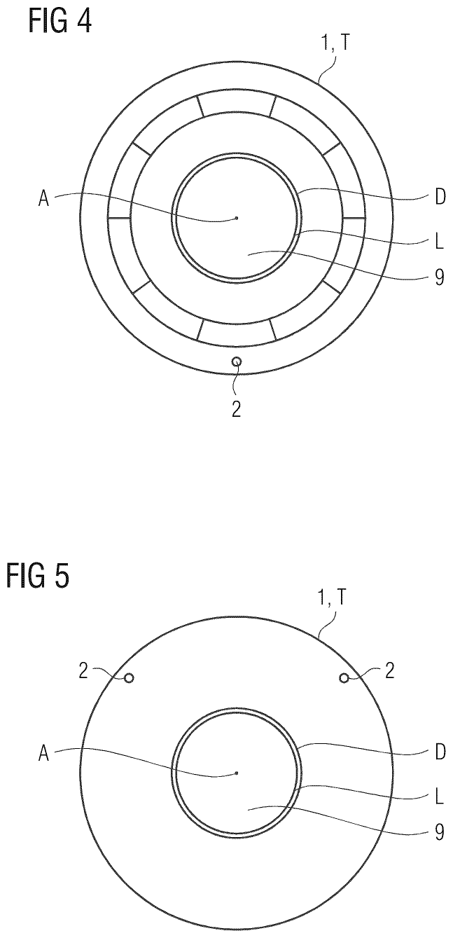

The FIGS. 4 and 5 show an example of the placement of three structure-borne sound sensors 2 on a gantry 1. In this case, the structure-borne sound sensors 2 are attached at set measuring points. The measuring points are set so as to be adapted for good comparability of structure-borne sound values captured by the structure-borne sound sensors 2 with the reference values of the airborne sound measurement. They also correspond to reference measuring points which were used to ascertain a reference curve. In this exemplary embodiment, one structure-borne sound sensor 2 is attached to the front side of the gantry 1 (FIG. 4) centrally at the bottom. Offset in each case by approximately 130° relative to the structure-borne sound sensor 2 on the front side, two further structure-borne sound sensors 2 are arranged on the rear side. The two structure-borne sound sensors 2 on the rear side are attached on opposite sides of the annular form of the gantry 1 relative to each other. The exact measuring positions can be adapted according to the gantry 1 that is used, in order to optimize the measurement of the structure-borne sound values.

The gantry 1 has a tunnel-shaped opening 9, said tunnel-shaped opening 9 extending along the axis of rotation A. The tunnel-shaped opening 9 is designed in such a way that that an examination object can be introduced into the tunnel-shaped opening 9 along the axis of rotation A for the purpose of examination via the computed tomography system. In particular, provision can be made for the examination object within the tunnel-shaped opening 9 to be stationary relative to the supporting structure T during the examination via the computed tomography system, while the bearing ring L rotates about the axis of rotation A relative to the supporting structure T, so that in particular the x-ray source and the x-ray detector of the computed tomography system rotate around the examination object.

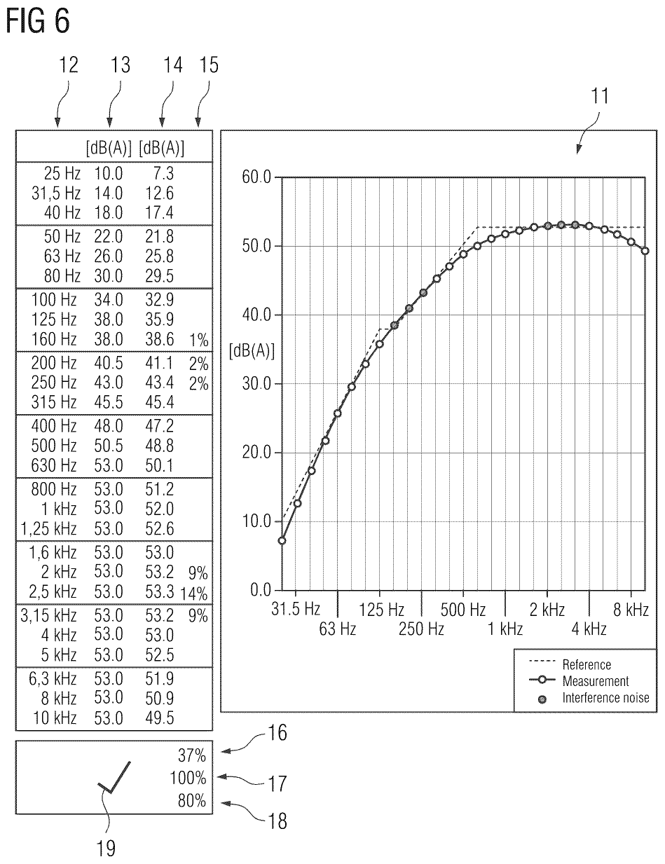

FIGS. 6 to 9 each show examples of an evaluation of structure-borne sound values. The structure-borne sound values are frequency-dependent mean velocity levels 14 in each case. On the left-hand side is a list of mean velocity levels 14, each of which is assigned to a third-octave center frequency 12. For each third-octave center frequency 12, a reference value 13 is furthermore assigned in each case to a reference curve. It is determined whether interference noise occurs during the rotation of the bearing ring L of the gantry 1, by comparing the measured mean velocity levels 14 with the reference values 13 of the reference curve. On the right-hand side of the table are the weighted instances of exceeding 15, which specify a relative interference noise level. The weighted instances of exceeding 15 are specified as a percentage of the interference noise tolerance threshold value 17, which defines the maximum permissible interference noise level (corresponding to 100%). The sum 16 of all weighted instances of exceeding 15 is specified below the table.

At 37%, this sum 16 in FIG. 6 lies below the interference noise tolerance threshold value 17 and also below a further interference noise threshold value 18, which is a threshold value for a warning here. On the right-hand side of FIGS. 6 to 9 is shown in each case a plot of the velocity level spectrum 11 together with a reference curve (broken marked). The mean velocity levels 14 (in dB(A)) relative to the third-octave center frequencies 12 are plotted here. The measuring points are identified by circles, where filled circles identify an instance of exceeding the reference curve. It can be seen in FIG. 6 that although some measuring points exceed the reference curve, the classification 19 is nonetheless positive because the sum 16 of all weighted instances of exceeding 15 lies both below the interference noise tolerance threshold value 17 of 100% and below the further interference noise threshold value 18 of 80%.

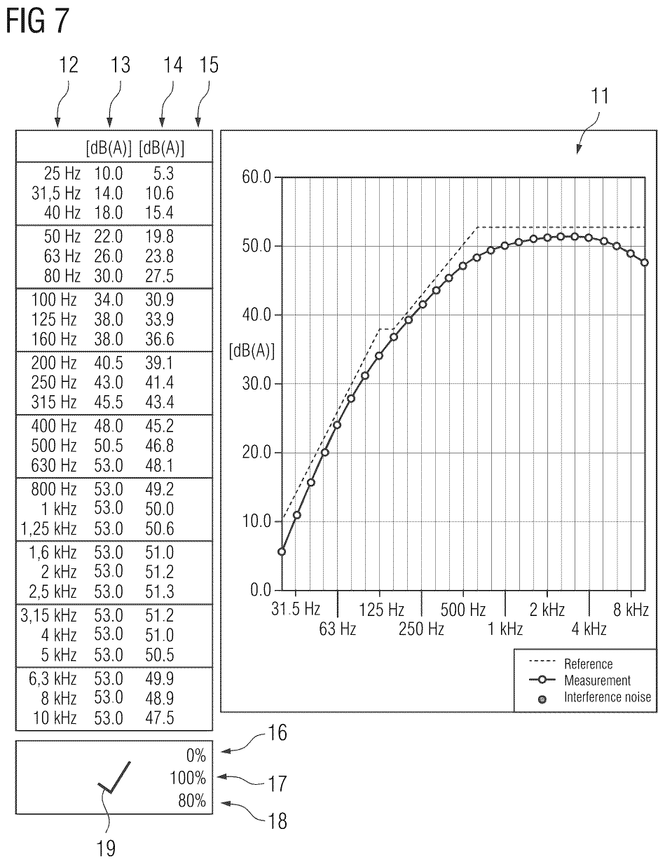

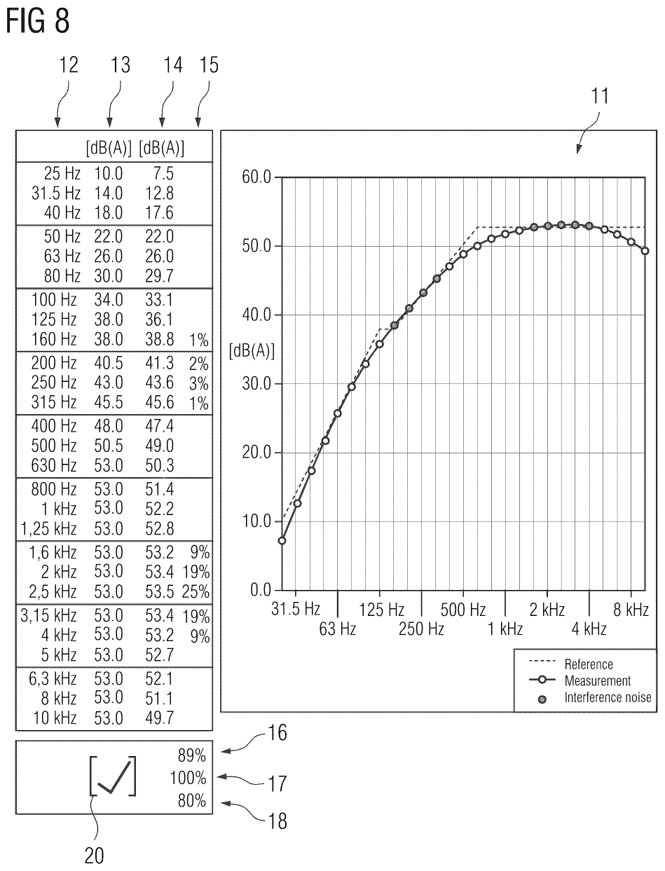

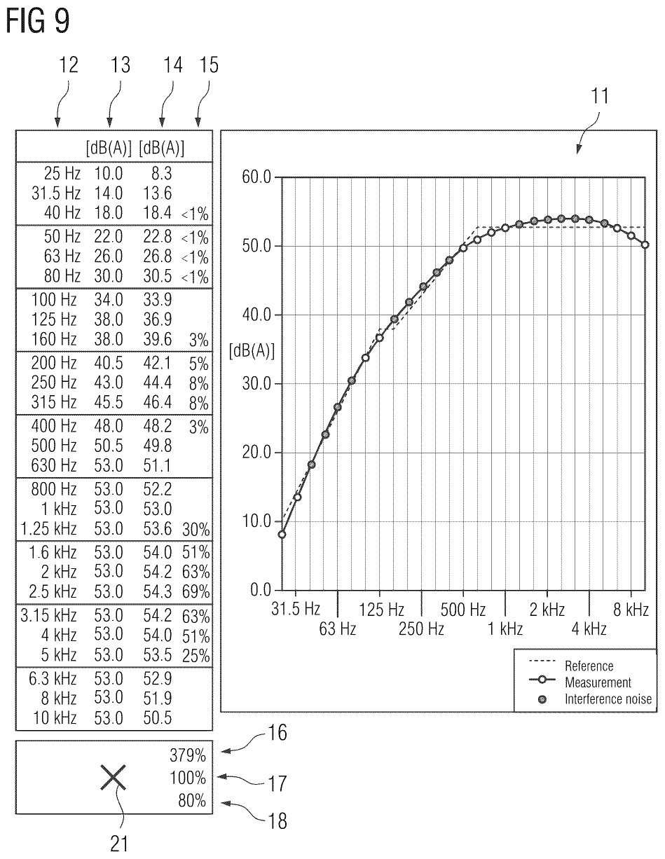

The classification 19 is likewise positive in FIG. 7. In the case of FIG. 7, indeed all measured mean velocity levels 14 lie below the reference values 13 of the reference curve, and therefore the sum 16 of all weighted instances of exceeding 15 is 0%. In FIG. 8, the sum 16 of all weighted instances of exceeding 15 lies above the further interference noise threshold value 18 but remains below the interference noise tolerance threshold value 17. The sum 16 of all weighted instances of exceeding 15 is 89% here, and is therefore less than 100% but greater than the 80% of the further interference noise threshold value. Therefore an intermediate classification 20 is indicated, signaling that the specified interference noise tolerance is almost reached. In the example of FIG. 9, however, the interference noise tolerance threshold value 17 is also clearly exceeded by the sum 16 of weighted instances of exceeding 15. The sum 16 of all weighted instances of exceeding 15 is 379% here, and is therefore greater than the 100% of the interference noise tolerance threshold value 17.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for checking a rotary bearing of a gantry of a computed tomography system, wherein the gantry has a supporting structure and the rotary bearing has a bearing ring, and wherein the bearing ring is mounted rotatably about an axis of rotation relative to the supporting structure, said method comprising:
rotating the bearing ring about the axis of rotation relative to the supporting structure, wherein at least two structure-borne sound sensors are attached to the gantry;
capturing structure-borne sound values via the at least two structure-borne sound sensors while the bearing ring rotates about the axis of rotation relative to the supporting structure; and
evaluating the structure-borne sound values to determine whether interference noise exceeding a specified interference noise tolerance occurs when the bearing ring rotates.

2. The method as claimed in claim 1, wherein the evaluating comprises breaking down the structure-borne sound values into a frequency spectrum.

3. The method as claimed in claim 1, wherein the evaluating comprises:
determining, in a frequency-dependent manner, whether the interference noise exceeding the specified interference noise tolerance occurs when the bearing ring is rotated.

4. The method as claimed in claim 1, wherein a velocity level spectrum is determined based on the structure-borne sound values.

5. The method as claimed in claim 4, wherein the evaluating comprises:
determining, based on the velocity level spectrum, whether the interference noise exceeding the specified interference noise tolerance occurs when the bearing ring is rotated.

6. The method as claimed in claim 5,
wherein mean velocity levels determined in the velocity level spectrum are assigned to specified frequency segments.

7. The method as claimed in claim 1, wherein the evaluating comprises:
determining, based on a correlation of the structure-borne sound values and with a reference curve, whether the interference noise exceeding the specified interference noise tolerance occurs when the bearing ring rotates.

8. The method as claimed in claim 7,
wherein individual instances of exceeding the reference curve are weighted according to an influence on a total interference noise,
wherein a sum of all weighted instances of exceeding is correlated with at least one interference noise threshold value, which includes an interference noise tolerance threshold value, such that a classification of the sum of all weighted instances of exceeding is performed, and
wherein an instance of exceeding the at least one interference noise threshold value is rated as an instance of exceeding the specified interference noise tolerance.

9. The method as claimed in claim 7,
wherein the at least two structure-borne sound sensors are attached at measuring points that correspond to reference measuring points used to ascertain the reference curve.

10. The method as claimed in claim 1, wherein the evaluating of the structure-borne sound values comprises at least one of
converting the structure-borne sound values into anticipated airborne sound values, or
correlating the structure-borne sound values with reference values of an airborne sound measurement.

11. The method as claimed in claim 10,
wherein the at least two structure-borne sound sensors are attached at measuring points having positions adapted to be comparable to the structure-borne sound values with the reference values of the airborne sound measurement.

12. The method as claimed in claim 1, wherein the at least two structure-borne sound sensors are attached to the supporting structure.

13. The method as claimed in claim 1, further comprising:
attaching the at least two structure-borne sound sensors to the gantry.

14. A method for evaluating structure-borne sound values for checking a rotary bearing of a gantry of a computed tomography system, said method comprising:
establishing a velocity level spectrum, wherein each velocity level value, among a plurality of velocity level values, is assigned to a frequency value;
correlating the plurality of velocity level values with reference values and registering instances of the reference values being exceeded;
weighting the instances of exceeding the reference values according to an associated influence on a total interference noise and summing the weighted instances of exceeding;
correlating the sum of the weighted instances of exceeding with at least one interference noise threshold value, which includes an interference noise tolerance threshold value; and
performing a classification of the sum of the weighted instances of exceeding, an instance of exceeding the interference noise tolerance threshold value being rated as an instance of exceeding a specified interference noise tolerance.

15. A non-transitory computer-readable storage medium storing computer-executable instructions that, when executed by a computer, cause said computer to perform the method as claimed in claim 14.

16. The method as claimed in claim 3, wherein the determining determines whether the interference noise exceeding the specified interference noise tolerance occurs when the bearing ring is rotated based on a frequency spectrum.

17. The method as claimed in claim 6, wherein the specified frequency segments are third-octave center frequencies.

18. The method as claimed in claim 7, wherein the correlation of the structure-borne sound values is in the form of a velocity level spectrum.

19. The method as claimed in claim 12, wherein the at least two structure-borne sound sensors are attached to a surface of the supporting structure.

20. The method as claimed in claim 13, wherein the attaching attaches the at least two structure-borne sound sensors to a supporting structure of the gantry.

21. The method as claimed in claim 14, wherein the plurality of velocity level values include mean velocity level values.

22. The method as claimed in claim 14, wherein to the frequency value is a third-octave center frequency.
